(19) United States
(12) Patent Application Publication
(10) Pub. No.: US 2025/0146898 A1
(43) Pub. Date: May 8, 2025

(54) Method and Device for Detecting a Water Leak in A Metallurgical Melting Furnace

(71) Applicant: CTH CONRADS TECHNOLOGIE UND HOLDING AG, Juelich (DE)

(72) Inventor: Hans Georg CONRADS, Hannover (DE)

(21) Appl. No.: 18/865,852

(22) PCT Filed: May 12, 2023
(86) PCT No.: PCT/DE2023/000033

§ 371 Date: Nov. 14, 2024

(30) Foreign Application Priority Data
May 16, 2022 (DE) 102022001718.0

(51) Int. Cl. G01M 3/26; F27B 3/28; F27D 9/00; G01N 21/31; G01N 33/00

(52) U.S. Cl. CPC G01M 3/26; F27B 3/28; F27D 9/00; G01N 21/31; G01N 33/0027; F27D 2009/0013

(57) ABSTRACT

The invention relates to a method for detecting a water leak in a metallurgical melting furnace. The invention also relates to a device for carrying out the method. A water leak in a cooling pipe or water pipe of a furnace vessel is detected by combining and correlating a time-resolved measurement of an exhaust gas parameter, such as the amount of water, at the exhaust gas outlet and a controlled pressure fluctuation, i.e. a variation in the pressure, of the cooling circuit. In the event of a leak, the variation in the water pressure causes a variation in the amount of water discharged into the furnace vessel, that is temporally correlated with the variation in the pressure of the cooling circuit. By determining the correlation of these signals, an evaluation device can determine a relationship based on a water leak.

The invention relates to a method for detecting a water leak in a metallurgical melting furnace, which has a furnace vessel constructed of walls consisting at least partially of water pipe walls through which cooling water flows, and to a device suitable for carrying out the method.

In a metallurgical melting furnace, such as an arc furnace, metals are melted and thereafter cast. This requires particularly high temperatures inside such a metallurgical melting furnace, so that the materials inside the furnace vessel must be able to withstand temperatures of up to 3500° C. Such temperatures pose a particular challenge for the systems used.

The individual components of a metallurgical melting furnace that come into contact with the liquid metal and the hot exhaust gases are therefore made either of refractory material or of water-cooled components. Some parts of the metallurgical melting furnace, in particular the furnace vessel, consist at least in sections of water tube walls with water tubes through which cooling water flows during operation. This reduces the risk of damage or destruction to the walls of the furnace vessel by the molten metal and the hot exhaust gases.

Such a metallurgical melting furnace having water tube walls is described in EP 2 601 469 B1. The water tube walls are designed in the form of fluid-cooled plates or panels and are particularly suitable for applications in metallurgical furnaces, in particular in electric arc furnaces for steel production. However, it is a common problem that the strong thermal loads acting on the water tube walls, in particular due to the large temperature differences, can cause leaks in the water tube walls. As a result, water flows into the metallurgical melting furnace.

In principle, water is also produced in the metallurgical melting furnace by combustion processes within the furnace vessel, for example by the combustion of added natural gas or hydrogen. Furthermore, at the beginning of the melting cycle, water is introduced by the metal components introduced into the furnace vessel. Another source for a water component originates from vertically installed graphite electrodes, along which water runs down into the furnace vessel for cooling. The water present in the furnace vessel evaporates and is discharged via the exhaust gas systems. Even in the event of a leak, however, the majority of the water present in the exhaust gas comes from the aforementioned sources.

The problem with the additional water entering the furnace vessel via water leaks is that it enters at a single point in an undefined and localized manner, so that the incoming water can collect in certain places. This is due to the fact that the metal components to be melted, such as scrap, are usually irregularly shaped and can have numerous pocket-shaped cavities in which the water remains at least partially and therefore does not completely evaporate. The quantity of water collected in liquid form in certain places can grow over time to form a continuously growing water bubble. When, as the metal scrap continues to heat up during the melting cycle and very hot, liquid metal comes into contact with such a water bubble, there is a risk of explosive evaporation. This can result in serious damage to the metallurgical melting furnace or even its destruction. In addition, metal masses that exit the metallurgical melting furnace and possibly splash around pose a serious danger to those working in the vicinity of the metallurgical melting furnace.

Various approaches to detecting such water leaks exist, although these approaches have so far been calculated using longer-term furnace balances. For example, DE 10 2009 051 931 A1 describes a method for the early detection of leaks in a cooling device for cooling a technical system, in particular a continuous casting plant. A controllable inlet-side valve and a controllable outlet-side valve are operated remotely and the deviations from expected pressures are determined. The pressure curve can also be examined for changes over time. However, due to the high process noise, such considerations cannot be used as a reliable indicator, as the dispersive effect of the entire process, in particular the input into the melting furnace, the evaporation during the process and also the discharge through the exhaust gas duct, is too difficult to determine and depends on a number of factors. The process noise is the result of process variables such as flow, pressure, temperature or water content caused by stochastic fluctuations and cross-effects. It should be noted that the respective quantities in complex engineering processes can never be measured in isolation, as they are usually influenced by other processes. For example, the water in the exhaust gas is fed by various sources whose timing and location cannot be clearly determined and that overlap in the measured signal. For example, switching a burner on and off and the spontaneous evaporation of water in a scrap batch regularly causes a sudden increase or change in the water content in the exhaust gas.

In contrast, water ingress from a leak is a gradual, uniform process that results in a comparatively low water ingress. The water ingress through a leak leads to a constant background of water vapor in the exhaust gas flowing out through the exhaust outlet, which makes it difficult or even impossible to clearly detect a water leak using state-of-the-art methods. This background signal of water ingress through the leak increases only very slowly, sometimes over weeks. Very precise material and furnace balances are required to be able to reliably detect such water content. Even the use of neural networks to evaluate the signal curves has so far proven to be very difficult, since such a network cannot be trained in a real furnace, as this would be too dangerous.

Even balancing the cooling water circuits, which examines the amount of water entering and exiting the water-cooling system per unit of time, does not produce reliable results. A water leak through which, for example, 3 m³ of water per hour escapes into the furnace vessel is too small to be reliably detected at a total flow of around 900 m³ per hour. In addition, water leaks can also occur outside the furnace in numerous other locations.

It is therefore the object of the invention to provide a simple and cost-effective technique for reliably detecting water leaks in a metallurgical melting furnace in spite of high process noise.

The object is achieved by a method and a device having the features according to the independent patent claims 1 and 8. Further developments are recited in the dependent claims.

The object is achieved in particular by a method for detecting a water leak in a metallurgical melting furnace. The method according to the invention is performed with a metallurgical melting furnace, an exhaust gas measuring device for measuring at least one exhaust gas parameter, a pressure regulator and an evaluation device.

The metallurgical melting furnace has a furnace vessel, the walls of which consist at least partially of water pipe walls through which cooling water flows, a lid and an exhaust gas outlet.

The exhaust gas measuring device is arranged in the direction of exhaust gas flow downstream of the exhaust gas outlet and is connected to the evaluation device for data transfer. Depending on the exact arrangement of the exhaust gas measuring device, detected leaks are assigned to different areas. According to an advantageous variant, the exhaust gas measuring device is arranged in close proximity downstream of the exhaust gas outlet, which advantageously prevents contributions from other water sources. Such an arrangement has the further advantage that leaks that only occur downstream of the furnace vessel, i.e. in the exhaust pipe, do not contribute. Since a leak in the water pipe walls of the furnace vessel poses an increased potential risk, it is advantageous to clarify whether the leak is indeed in the furnace vessel. A leak in the exhaust duct, on the other hand, is harmless in relation to its risk potential.

Furthermore, the evaluation device is also connected to the pressure regulator for data transfer. The pressure regulator can set and regulate the pressure of the cooling water in the water pipe walls.

The process includes the following steps:
a) Subjecting the cooling water to a pressure fluctuation, whereby a pressure fluctuation is a consequence of time-limited pressure deviations from an average pressure, produced by the pressure regulator;
b) Measuring, with the exhaust gas measuring device, the course of an exhaust gas parameter, i.e. determining the measured values over time;
c) Determining with the evaluation device the correlation between the course of the pressure fluctuation of the cooling water and the course of the exhaust gas parameter. A correlation function is used for this purpose;
d) Outputting the correlation by the evaluation device.

The result of the correlation can be processed within the evaluation device after each evaluation cycle so that a signal is only outputted when certain conditions are met, or the correlation is outputted after each evaluation cycle.

In the context of the invention, cooling water refers to a fluid that can be used in a cooling device to cool a technical system. In particular, the fluid can be water. The method according to the invention can also be carried out with other fluids, in particular with other cooling liquids.

Preferably, the evaluation device delivers an output signal, for example a warning signal, when predetermined criteria are met.

Preferably, in order to determine the correlation according to step c), the course of the pressure fluctuation is forwarded to the evaluation device or entered into the evaluation device. Furthermore, the result of the measurement according to step b) is forwarded to the evaluation device.

In the context of the invention, the course of a parameter is a series of values of this parameter which were determined sequentially. In other words, the temporal evolution of this parameter is shown.

In the context of the invention, subjecting the cooling water to a pressure fluctuation means generating a pressure fluctuation, in particular generating a sequence of predetermined, time-limited pressure deviations, i.e. a sequence of predetermined, time-limited increases and/or decreases in the pressure, in particular in the nominal pressure, of the cooling water, whereby the pressure can deviate upwards and/or downwards from an average pressure. The pressure is the pressure of the cooling water or the cooling liquid in the cooling water pipes of the water pipe walls.

In the context of the invention, a pressure fluctuation refers to a sequence of time-limited pressure deviations from an average pressure. Several consecutive pressure deviations follow a certain predetermined pattern.

Such a pressure deviation has a certain duration and a certain deviation value, i.e. an amplitude and a deviation direction. In this sense, the deviation value describes the level of the deviation from an average pressure, whereby a deviation value can be both positive and negative. Furthermore, the pressure deviations can also have a certain temporal interval between each other.

In the event of a leak, an increase or decrease in the pressure of the cooling water leads to an increase or decrease in the amount of cooling water entering the furnace through the leak, i.e. the leakage amount. This creates an image of the pattern of the predetermined pressure fluctuation impressed on the cooling water, i.e. the sequence of predetermined pressure deviations, also in the exhaust gas in the form of a change in at least one exhaust gas parameter, such as the water concentration. This change in the exhaust gas parameter occurs in accordance with the predetermined pressure fluctuation. A correlation between the predetermined pressure fluctuation in the cooling water and the fluctuations in the values of the exhaust gas parameter can be used to determine whether there is a leak in the cooling system.

In order to obtain a pressure fluctuation, i.e. a sequence of pressure deviations, which can preferably be used for a correlation, at least one pressure deviation parameter selected from duration, deviation value and distance is adjusted from one pressure fluctuation to the next. In the case of a sequence of pressure deviations, i.e. in the case of a pressure fluctuation, the duration and/or the deviation value and/or the distance of the pressure deviation is varied.

In a sequence of pressure fluctuations, either just one pressure deviation parameter, for example just the deviation value or just the duration of the pressure deviations, or at least two of the pressure deviation parameters, for example, both the duration and the deviation value, can be varied. This produces a characteristic pattern, which is explored via a correlation in at least one of the exhaust gas parameters. Since the pipes also cause a pressure loss, the duration, also referred to as the temporal duration, is an important criterion for the correlation and the evaluation.

According to an advantageous embodiment, the sequence of pressure deviation parameters is determined using a random generator, also referred to as a random number generator. An irregular sequence of pressure deviations, and thus also an irregular pressure fluctuation, can be realized using a random generator. The sequence of pressure deviations is determined by a value generated by the random generator between a minimum and a maximum pressure deviation parameter, for example a value generated by the random generator between a minimum and a maximum time period.

Certain smallest units can also be defined for this purpose, so that a pressure deviation parameter can be randomly selected from, for example, 3, 5, 8 or 10 values. A deterministic random number generator can be used for this purpose.

Alternatively, a non-deterministic or hybrid random number generator can be used.

A coincidence of irregular changes in one of the exhaust gas parameters with other effects, more frequently associated with periodic pressure fluctuations, for example, can thus be prevented or the probability can at least be greatly reduced.

For example, a program can be used to generate a sequence of 1-second pulses with varying deviation values. The number of pulses, each having the same deviation value, is determined by a randomly generated number between a minimum duration and a maximum duration, while the deviation value itself is determined by a randomly generated number between a minimum deviation value and a maximum deviation value. An additional change in amplitude up to the maximum value is possible.

According to one possible design, the pressure fluctuation can be generated continuously, advantageously allowing leaks to be detected at any time.

Optionally, certain time periods of, for example, 1800 s are specified, which are then fed to a correlation. Preferably, the patterns do not repeat after this time period, and a new pattern is generated instead, which is then used to perform a further correlation. In this way, a positive result can be advantageously verified or refuted in a two-stage process. This improves the reliability of the process.

An exhaust gas parameter is a parameter of the exhaust gas, for example the proportion or the amount of water contained in the exhaust gas or the pressure of the exhaust gas or the temperature of the exhaust gas. The characteristic pattern caused by the pressurization can be advantageously read from different parameters or process variables. Thus, different exhaust gas parameters can advantageously be used for the correlation measurement and serve as a reliable indicator of the leak.

In the context of the invention, a correlation is to be understood to be a relationship between two signal sequences, in this case the course of the exhaust gas parameter and the pressure fluctuation over time. It can be determined from the correlation whether a connection between the courses can be identified and consequently a connection between the courses exists. If certain values and limits are set depending on the selected evaluation method and correlation function, the result of the correlation can be used to decide whether a leak is present. Since this is a statistical process, the limits can be selected depending on the desired and necessary probability of correct or incorrect warning signals.

An advantage of this method is that a signal can be detected even if there are other possibilities for water entering the furnace vessel. A small correlated fluctuation in the amount of water measured at the exhaust gas outlet due to a leak can be reliably detected even if the fluctuation in the amount of water due to other processes is very large.

Even if the amounts of water introduced into the furnace process by the other sources, particularly in conjunction with their temporal uncertainty, cause the signal to be detected to be very small, the method according to the invention can still be used to perform an evaluation. Advantageously, by correlating the pressure fluctuation of the cooling water in the water pipe walls with the measured values of the water quantity in the exhaust gas, leaks in the water pipe walls of the furnace vessel can be reliably detected.

It should be taken into account that even when a portion of the water entering the furnace vessel through a leak collects in liquid form at one location, part of the water will always evaporate. The method according to the invention is based on the fact that the evaporated part of the water, which flows out through the exhaust gas outlet, impacts the signal of the exhaust gas measuring device.

According to an advantageous embodiment of the method, cooling water flows through at least two different sections within the water pipe walls, which are subjected to different pressure fluctuations according to step a), and a correlation is carried out according to step c) for each of the sections. The frequency and/or amplitude and/or deviation value of these pressure fluctuations can be varied, for example.

According to an advantageous variant, for each section there is provided an auxiliary line, which is connected to this section. The different sections preferably form different cooling circuits. With different cooling circuits, different pressure curve patterns can be imposed in each circuit. This can happen in the circuits both at different times, i.e. sequentially, as well as simultaneously. If the patterns are different, the circuit that has a leak will be detected by the corresponding correlation. Depending on the characteristics of the exhaust gas parameter and the correlation determined therefrom, the correlation corresponds to the characteristics of the pressurization in one or more sections. This can advantageously be used to determine in which section or sections the leak is present.

According to one possible embodiment, each section of a water pipe wall through which cooling water flows has a separate pressure regulator, or a unit assigned to the section and configured to apply a pressure fluctuation to the pressure within the respective section.

According to one possible embodiment, valves are arranged within the water pipe walls between the sections. These enable different characteristics of pressurization even in connected sections or interconnected cooling circuits. Installing valves divides a cooling circuit into different sections or zones, which can then be checked separately for leaks. Preferably, the exhaust gas parameter measured in step b) is the gas velocity and/or the water content and/or the water quantity and/or the pressure of the exhaust gas and/or the temperature of the exhaust gas and/or carbon monoxide (CO) or carbon dioxide (CO₂) or methane (CH₄) or sulfur dioxide (SO₂). These exhaust gas parameters are influenced by leaking water and are therefore suitable as parameters for a correlation measurement. Preferably, one of the exhaust gas parameters evaluated is the water content and/or the water quantity, since these have a particularly strong correlation with incoming water. This is particularly advantageous for the signal-to-noise ratio.

However, if other exhaust gas parameters have already been determined for other reasons, then it may be appropriate and efficient to also use these exhaust gas parameters as a basis for such correlation.

Optionally, several exhaust gas parameters, in particular their course, are measured and checked using a correlation. The signal-to-noise ratio can thus be further improved. This advantageously makes it possible to achieve more precise and reliable results.

For the variant of the method in which the measured exhaust gas parameter is the gas velocity, the following calculation example shows the advantages of the method.

A typical leak begins with approximately 1-2 l of water per minute entering the furnace vessel. 1 l of water causes a volume of approximately 7 m³ of water vapor under normal pressure. However, if the temperature is above 1000° C., this results in a volume of approximately 420 m³ of water vapor. With an outlet diameter of 4 m and a maximum flow velocity of 25 m per second, this results in a maximum volume flow of 314 m³ of water vapor per second. Depending on the flow profile in the outlet, the actual volume flow is approximately 50% of the maximum value, i.e. approximately 157 m³ of water vapor per second or 565,000 m³ of water vapor per hour. The volume flow of water vapor introduced by a leak of 1 l of water per minute is therefore only 0.075% of the total volume flow of water vapor. According to the Bernoulli equation, the flow of incompressible liquids changes proportionally to the square root of the pressure. Doubling the pressure therefore leads to an increase in the amount of water leaking by a factor of approx. 1.4, which for the example calculation results in an increase in the volume flow of water vapor to approx. 595 m³ of water vapor per second or 0.105% of the total volume flow of water vapor.

Due to typical process noise, such a change cannot be reliably detected either statistically or in a one-off manner. However, using frequently correlated repetition, such change becomes clearly apparent and can serve as a reliable measure of leak detection.

For the variant of the method, the sensitivity of the method can be further increased, i.e. improved, by measuring the water content directly with the exhaust gas measuring device. It is not necessary to measure the change in the overall composition of the exhaust gases, but only the relative change in the water.

Other processes causing water to enter the furnace vessel include the gas burner or the water cooling of the electrodes, wherein water runs past the electrodes into the furnace vessel where it immediately evaporates. This causes around 100 l/min of water to enter. In addition, the burner introduces up to 2000 m³/h of methane into the furnace vessel.

The scrap batch can also cause a significantly larger amount of water to be produced in a short period of time, which however quickly disappears again during the melting cycle. However, water ingress from such a scrap batch is irrelevant for the correlation, as this water ingress has no connection with the pressure fluctuation in the cooling water.

Due to the improved signal-to-noise ratio, direct detection of the amount of water at the exhaust outlet improves the possibilities of leak detection compared to measuring the flow rate of the total exhaust gas quantity at the exhaust outlet. This leads to a reduction of the required pressure fluctuation, which minimizes the mechanical load on the components. The cooling water is typically fed into the cooling water circuit at around 25-35° C. and thus experiences a temperature increase of around 15° C. at a nominal throughput. Since an increase in pressure puts more strain on the components in the long term, while a reduction in pressure leads to an increase in the water outlet temperature, a small fluctuation around a suitable water pressure is desirable. Since a fluctuation of just 10% in pressure only causes a change in the water discharge of slightly less than 5%, an optimized evaluation is necessary.

According to an advantageous variant, the pressure fluctuation is irregular, i.e. the pressure fluctuation applied according to step a) follows a predetermined, irregular pattern. The cooling water is subjected to a predetermined, irregular sequence of pressure deviations according to step a). This advantageously produces a distinct temporal signal at the exhaust gas outlet, which has no periodicity and contains a strong signal entropy over a longer period of time.

A non-periodic pressure fluctuation also repeats itself after a certain time unit. The time unit after which such a pressure fluctuation is repeated is preferably greater than half an hour.

The measuring cycles do not necessarily have to be particularly short in order to reduce the risk. It should be noted that an accident due to explosive evaporation processes within the furnace vessel does not necessarily occur in every melting cycle. A water leak can remain undetected for a long time and grow, with an increasing amount of water entering the furnace vessel. However, if a corresponding arrangement of scrap or other batch-related changes in the geometry in the furnace vessel cause water to accumulate in the lower area of the furnace vessel, this can lead to a spontaneous explosion.

The time constants must be selected so that the relative slope of the actual change in quantity of water can still be reliably detected with a correlation. The signals for the change in water pressure can be in the time frame from several 10 s to several minutes. Several signal trains from various melting processes can thus be combined into one correlatable signal train.

According to an advantageous variant of the method, filtering can be performed in the frequency spectrum commensurate with the pressure fluctuation of the water pressure according to step a), whereby the signal of the exhaust gas measuring device is adapted. This advantageously improves the signal-to-noise ratio.

The cooling water preferably passes through the cooling pipes at a pressure of 6 bar. Preferably, a pressure fluctuation introduced by the pressure regulator according to step a) provides a variation of the pressure by +2 bar upwards and −2 bar downwards. A suitable pressure fluctuation is preferably in a range of 10% to 50% of the pressure, i.e. usually the average value of the pressure, at which the fluctuation occurs.

By varying the water pressure in this way, in the event of a leak, the amount of water escaping per unit of time would be slightly changed in a controlled manner due to the water pressure.

The application of a pressure fluctuation or preferably an irregular sequence of pressure deviations to the cooling water is implemented by using a pressure regulator described herein, which is also referred to as a pressure control device.

One possible embodiment of the method provides that the correlation according to step c) is a cross-correlation. If a clear maximum of the cross-correlation is obtained, it can be assumed that a water leak is present. The cross-correlation has then detected the time pattern of the water pressure change in the exhaust gas signal, i.e. in the signal from the exhaust gas measuring device.

Advantageously, by using cross-correlation, a useful signal can be obtained even from a severely corrupted or noisy signal. It is important to use a sufficiently long signal so that the signal contains enough statistical information.

The reliable formation of a maximum of the cross-correlation enables reliable detection of a water leak. Interference signals from other water incursions, which are subject to a different temporal dynamic, are suppressed so strongly by a cross-correlation that even small water leaks can be detected. Small water leaks in the context of the invention are leaks with a water discharge of 1-10 liters per minute or 1 to 5 liters per minute or 1 to 2 liters per minute. Preferably, the method according to the invention can detect a leak which is at least 3 liters per minute, preferably 1 liter per minute.

According to an advantageous embodiment of the method, the measurement according to step b) is carried out using spectroscopy. In the context of the invention, spectroscopy is a measuring method that uses optical principles while considering the individual wavelengths of the evaluated signal. A laser-based emission measurement or a laser-based absorption measurement is preferably used.

Because optical measuring systems working on the principle of spectroscopy are employed, the composition of the exhaust gases can be determined by measuring emission or absorption spectra in specific wavelength ranges. Such an optical measuring system is preferably arranged in or on the exhaust pipe. Preferably, online systems or extractive systems can be used, which suction off a partial flow of the exhaust gas through a water-cooled lance. With optical measuring systems, a time-resolved analysis of the exhaust gas composition can advantageously be performed. The amount of water at the exhaust outlet can therefore advantageously be measured directly.

An advantageous embodiment of the method provides that the metallurgical melting furnace is designed as an arc furnace with electrodes as a heating device.

A further aspect of the invention relates to a device for carrying out the method according to the invention, including a metallurgical melting furnace, preferably designed as an arc furnace, an exhaust gas measuring device for measuring at least one exhaust gas parameter according to step b), a pressure regulator for applying a pressure fluctuation to the pressure of the cooling water according to step a), and an evaluation device connected to the pressure regulator for data transfer for carrying out the correlation according to step c).

The metallurgical melting furnace includes a furnace vessel, the walls of which are constructed at least partially of water pipe walls through which cooling water flows, and an exhaust gas outlet. The exhaust gas measuring device is arranged downstream of the exhaust gas outlet in the exhaust gas flow direction and is connected to the evaluation device for data transfer.

Preferably, the metallurgical melting furnace is designed as an arc furnace with electrodes as heating device.

Preferably, the metallurgical melting furnace has a lid, particularly preferably a pivotable lid.

A pressure fluctuation or preferably an irregular sequence of pressure deviations is applied to the cooling water by way of a pressure regulator. In the context of the invention, a pressure regulator is a device that can control and/or regulate the pressure of the cooling medium flowing through the water pipe walls.

On a main line, a pressure regulator usually has a controllable and/or adjustable main pump for conveying the cooling water through the water pipes of the water pipe walls. According to one possible variant, a change in pressure can be achieved by controlling and/or regulating the main pump. The main pump is preferably designed and arranged in such a way that controlling the pump leads directly to a change in the pressure of the coolant and thus directly to the cooling water being subjected to a pressure fluctuation. A pattern of pressure fluctuation is thus achieved by changing or adjusting the delivery capacity of the main pump. This is a simple variant for implementing the application of a pressure fluctuation, for which no additional components need to be installed.

In an advantageous embodiment, the pressure regulator is designed as a pressure control device which, in addition to a main pump on a main cooling water line, has an auxiliary line connected to the cooling circuit, in particular to the water pipe walls, as a bypass conducting a portion of the cooling water flow. At least one, optionally several controllable and/or adjustable valves are provided on the auxiliary line, which vary the flow through the auxiliary line and thus also the flow through the main line. This influences the pressure in the main cooling line and can thus generate a pressure fluctuation in the cooling water. By generating the pressure fluctuation by varying a volume flow in an auxiliary line, the main pump advantageously needs to generate less frequent and less severe pressure changes. This advantageously protects the main pump from premature wear.

According to a possible design of a pressure regulator, the pressure regulator is designed as a pressure control device, wherein a change in the volume in the cooling water circuit generates the pressure fluctuation. Preferably, a piston constructed as a stamp is movably arranged on a volume change area of the water pipe walls, with a movement of the piston varying the volume and thus the pressure within the water pipe walls. Here too, it is advantageous to dispense with generating the pressure fluctuation with the main pump, so that it is protected against wear.

According to an advantageous embodiment, the pressure regulator has an auxiliary pump in addition to a main pump for generating a cooling water flow. The auxiliary pump is designed to be controllable and/or adjustable in such a way that an additional volume flow is generated and the pressure fluctuation can thus be generated by the auxiliary pump. The auxiliary pump is preferably smaller in size than the main pump. The auxiliary pump preferably has a pumping capacity of 5% to 20% of the main pump. A smaller auxiliary pump can advantageously be controlled more quickly and precisely, which improves the efficiency of the process.

The pressure regulator is preferably designed as a pressure control device which, in addition to a main pump on a main cooling water line, has an auxiliary line connected to the cooling circuit as a bypass allowing a portion of the cooling water to flow, wherein an auxiliary pump controls and/or regulates the cooling water flow through the auxiliary line. Generating the pressure fluctuation with an auxiliary pump advantageously leads to a greater steepness of the pressure deviations compared to pressure deviations that can be generated by means of the main pump, and also means that the main pump, which is usually larger, has to impart less frequent and less severe pressure changes on the larger portion of the cooling water flow. This advantageously protects the main pump from premature wear. The auxiliary line is preferably dimensioned such that a flow of 5% to 20% of the cooling water is generated through this bypass.

According to an alternative design, an auxiliary pump is arranged directly on the main cooling water line, thereby generating an additional volume flow with the auxiliary pump and thus protecting the main pump from wear caused by frequent pressure changes.

Particularly preferably, the water pipe walls through which cooling water flows include at least two sections, which can be subjected to different pressure fluctuations by means of several pressure regulators. Valves can be arranged between the sections.

According to the concept, a water leak in a cooling pipe or water pipe of a furnace vessel is detected by combining and correlating a time-resolved measurement of an exhaust gas parameter, such as the amount of water, at the exhaust gas outlet and a controlled pressure fluctuation, i.e. a variation in the pressure, of the cooling circuit. In the event of a leak, the variation in water pressure causes a variation in the amount of water discharged into the furnace vessel, which is correlated in time with the variation in the pressure of the cooling circuit. By determining the correlation of these signals, an evaluation device can determine a connection based on a water leak. An advantageous embodiment provides various cooling circuits on which different pressure curve patterns can be imparted. In addition to merely detecting the leak, this makes it also possible to determine the section, i.e. the cooling circuit, in which the leak occurs. Advantageously, the location of the leak can thus be narrowed down and more efficiently determined.

Further details, features and advantages of embodiments of the invention can be inferred from the following description of embodiments with reference to the appended drawings, which show in:

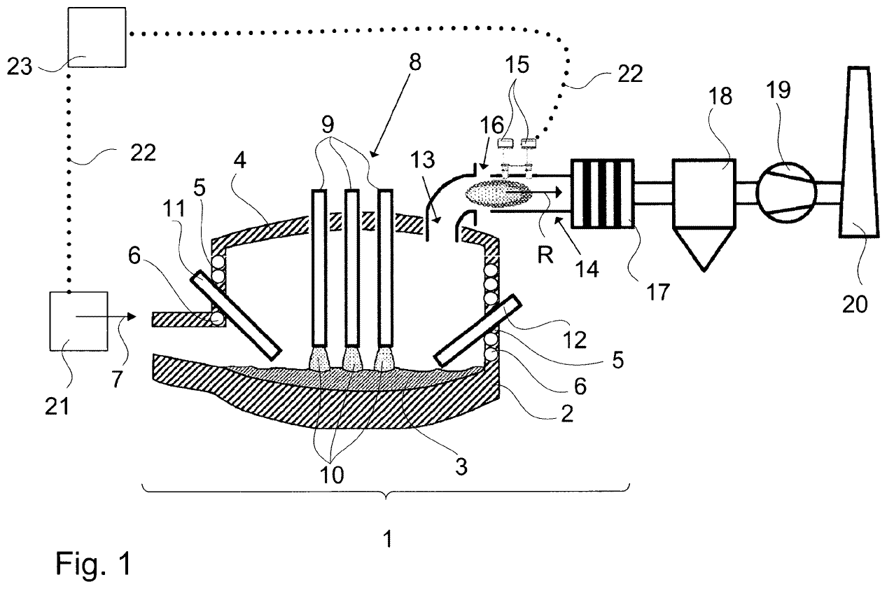

FIG. 1: a metallurgical melting furnace, and

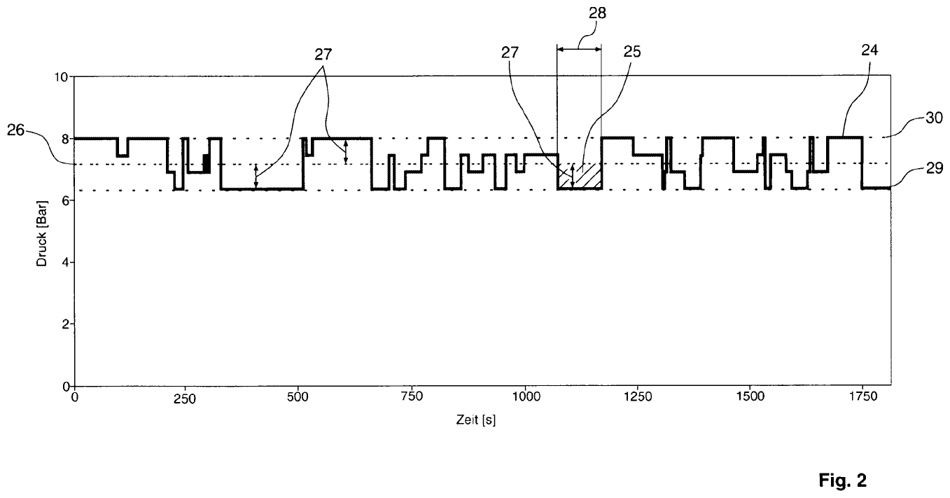

FIG. 2: a pressure fluctuation with an irregular pattern.

FIG. 1 shows a metallurgical melting furnace 1 for melting metal, which has a furnace vessel 2 with a metal melt 3 and a pivoting lid 4. Water pipe walls 5, i.e. walls which are traversed by water pipes 6 through which water flows, are arranged In the wall area of the furnace vessel 2. Water cooling is achieved by the cooling water 7 flowing through the water pipes 6 of the water pipe walls 5. This protects the furnace vessel 2 from damage caused by the high temperatures of the metal melt 3.

A heating device 6 having three electrodes 9 is arranged on the furnace vessel. The electrodes 9 protruding into the furnace vessel 2 are preferably designed for supply with three-phase alternating current. The electrodes 9 generate arcs 10, with the heat from the arcs 10 being used to melt the metal in the furnace vessel 2. A gas burner 11 and an oxygen supply element 12 designed as an oxygen lance 12 are also arranged on the furnace vessel 2.

The exhaust gases produced by the combustion and melting processes within the furnace vessel 2 are conducted through an exhaust gas outlet 13 into an exhaust gas pipe 14. An exhaust gas measuring device 15 for determining the exhaust gas parameters and monitoring the produced exhaust gases is also arranged on this exhaust gas pipe 14 connected to the exhaust gas outlet 13. The exhaust gas flows through the exhaust gas outlet 13, passes through an air supply opening 14, which is designed here as an air supply ring 16, and then flows past at least one exhaust gas measuring device 15.

Alternatively, the exhaust gas measuring device 15 is arranged in the exhaust gas flow direction R upstream of the air supply opening 16. However, arranging the exhaust gas measuring devices 15 downstream of the air supply opening 16 has structural advantages, since this area of the exhaust pipe 14 can be thermally separated from the furnace vessel 2. This reduces the thermal loads on the exhaust gas measuring device 15.

A cooler 17 for cooling the exhaust gas flow and a filter 18 for separating solid particles from the exhaust gas are arranged on the exhaust pipe 14 further downstream of the air supply device. The exhaust gas is then guided through the exhaust fan 19 and into the chimney 20.

To detect a leak in the water pipe walls 5, a pressure regulator 21 imparts the cooling water 7 flowing into the water pipes 6 of the water pipe walls 5 a pressure fluctuation, in particular a sequence of pressure deviations. The pressure fluctuation of the cooling water 7, i.e. in particular the course of the pressure fluctuation or the pattern of the sequence of pressure deviations of the cooling water 7, is compared with the course of an exhaust gas parameter measured at the exhaust gas measuring device 15. For this purpose, the signal of the exhaust gas parameters is forwarded to an evaluation device 23 via a data cable 22. The course of the pressure fluctuation of the cooling water 7 is also forwarded by the pressure regulator 21 to the evaluation device 23 via a data cable 22. The evaluation device 23 determines the correlation of the pressure fluctuation, i.e. the sequence of predetermined pressure deviations, also referred to as the pressure curve, of the cooling water 7 with the curve of the exhaust gas parameter measured at the exhaust gas measuring device 15, whereby relationships are recognized and reliable leak detection is possible.

FIG. 2 shows a pressure curve 24 to which a pressure fluctuation with an irregular pattern is imparted. The pressure fluctuation is formed by several consecutive pressure deviations 25, i.e. deviations from an average pressure 26. The pressure curve 24 thus assumes a specific pattern.

Each pressure deviation 25 has a deviation value 25 and a temporal duration 28, also referred to as duration 28. Using a random generator, an irregular sequence can be realized in which a value generated by the random generator between a minimum 29 and a maximum 30, for example between a minimum and a maximum deviation value and/or a value generated by the random generator between a minimum and a maximum duration, determines the sequence of pressure deviations 25. Specific smallest units can also be defined for this purpose, so that a random selection is made from, for example, 8 or 10 values.

The pressure deviations 25 shown here are not spaced apart from one another. However, it is also possible to provide spaces between the pressure deviations 25 without changing the average value, i.e. the average pressure 26. These spaces can also be varied. According to a simple design of the method, only the spaces between the pressure deviations 25 may be varied.

### LIST OF REFERENCE SYMBOLS

1 metallurgical melting furnace, arc furnace
2 furnace vessel
3 metal melt
4 lid
5 water pipe wall
6 water pipe
7 cooling water
8 heating device
9 electrode
10 arc
11 gas burner
12 oxygen supply element, oxygen lance
13 exhaust outlet
14 exhaust pipe
15 exhaust gas measuring device
16 air supply opening, air supply ring
17 cooler
18 filter
19 exhaust fan
20 chimney
21 pressure regulator
22 data cable
23 evaluation device
24 pressure
25 pressure deviation
26 average pressure
27 deviation value
28 duration
29 minimum
30 maximum
R exhaust gas flow direction

1. A method for detecting a water leak in a metallurgical melting furnace (1) by means of:
a metallurgical melting furnace (1), comprising a furnace vessel (2), the walls of which are composed at least partially of water pipe walls (5) through which cooling water (7) flows, and an exhaust gas outlet (13),
an exhaust gas measuring device (15) arranged in the exhaust gas flow direction (R) downstream of the exhaust gas outlet (13) for measuring at least one exhaust gas parameter,
a pressure regulator (21) for adjusting the pressure of the cooling water (7) in the water pipe walls (5), and
an evaluation device (23) connected to the exhaust gas measuring device (15) and the pressure regulator (21) for data transfer,
comprising the following steps:
a) applying with the pressure regulator (21) a pressure fluctuation to the cooling water (7), wherein a pressure fluctuation is a sequence of time-limited pressure deviations from an average pressure,
b) measuring with the exhaust gas measuring device (15) the course of an exhaust gas parameter,
c) determining with the evaluation device (23) the correlation of the course of the pressure fluctuation of the cooling water (7) with the course of the exhaust gas parameter, and
d) outputting of the correlation with the evaluation device (23).

2. The method according to claim 1, wherein the pressure fluctuation applied according to step a) follows a predetermined, irregular pattern.

3. The method according to claim 2, wherein a random generator is used to create an irregular sequence of pressure deviations (25).

4. The method according to claim 1, wherein cooling water (7) flows within the water pipe walls (5) at least through two different sections which are subjected to different pressure fluctuations according to step a), and wherein a correlation according to step c) is performed for each of the sections.

5. The method according to claim 4, wherein valves are arranged within the water pipe walls (5) between the sections.

6. The method according to claim 1, wherein the exhaust gas parameter measured in step b) is the gas velocity and/or the water content and/or the water quantity.

7. The method according to claim 1, wherein the pressure fluctuation in step a) is non-periodic.

8. The method according to claim 1, wherein the correlation according to step c) is a cross-correlation.

9. The method according to claim 1, wherein the measurement according to step b) is carried out by means of spectroscopy.

10. The method according to claim 9, wherein the measurement is a laser-based emission measurement or a laser-based absorption measurement.

11. The method according to claim 1, wherein the metallurgical melting furnace (1) is constructed as an arc furnace (1) with electrodes (9) as a heating device (8).

12. A device for carrying out the method according to claim 1, the device comprising
a metallurgical melting furnace (1) comprising a furnace vessel (2), the walls of which consist at least partially of water pipe walls (5) through which cooling water (7) flows, and an exhaust gas outlet (13) and
an exhaust gas measuring device (15) arranged in the exhaust gas flow direction (R) downstream of the exhaust gas outlet (13) for measuring at least one exhaust gas parameter according to step b),
a pressure regulator (21) for imparting on the pressure of the cooling water (7) a pressure fluctuation according to step a), and
an evaluation device (23) connected to the pressure regulator (21) and to the exhaust gas measuring device (15) for carrying out the correlation according to step c).

13. The device of claim 12, wherein the pressure regulator has a main pump and an auxiliary pump, wherein the auxiliary pump is designed to be controllable and/or adjustable in such a way that an additional volume flow generated by the auxiliary pump produces the pressure fluctuation.

14. The device of claim 12, wherein the pressure regulator (21) has an auxiliary line connected to the water pipe walls, wherein a valve is designed and arranged on the auxiliary line such that the flow through this auxiliary line can be controlled and/or regulated by the valve.

15. The device of claim 14, wherein the pressure regulator (21) has a volume change region on which a piston is designed and arranged such that a volume change generated by the movement of the piston influences the pressure in the water pipe walls.
